(19) United States
(12) Patent Application Publication
(10) Pub. No.: US 2023/0346771 A1
(43) Pub. Date: Nov. 2, 2023

(54) INHIBITORS OF ENPP1 AND MODULATION OF BONE GROWTH

(71) Applicant: Petragen, Inc., Davie, FL (US)

(72) Inventor: David Kolb, Davie, FL (US)

(21) Appl. No.: 18/308,288

(22) Filed: Apr. 27, 2023

Related U.S. Application Data
(60) Provisional application No. 63/336,630, filed on Apr. 29, 2022.

(51) Int. Cl. A61K 31/47; A61P 19/08

(52) U.S. Cl. CPC A61K 31/47; A61P 19/08

(57) ABSTRACT

Described are compositions of inhibitors of ectonucleotide pyrophosphatase/phosphodiesterase 1 (ENPP1) or pharmaceutically acceptable salts of the ENPP1 inhibitors, and methods of use thereof. The compositions are generally used to promote bone mineralization, bone growth, or both, mediated by ENPP1, particularly the alveolar bone.

### CROSS-REFERENCE TO RELATED APPLICATIONS

This application claims the benefit of and priority to U.S. Provisional Application No. 63/336,630 filed Apr. 29, 2022, which is hereby incorporated by reference in its entirety.

### FIELD OF THE INVENTION

This invention is generally in the field of ectonucleotide pyrophosphatase/phosphodiesterase 1 (ENPP1) inhibition, particularly compositions containing an ENPP1 inhibitor or pharmaceutically acceptable salt thereof, and a pharmaceutically acceptable carrier, for modulating bone growth in a subject.

### BACKGROUND OF THE INVENTION

ENPP1 is a type II transmembrane glycoprotein containing two identical disulfide-bonded subunits, and possesses nucleotide pyrophosphatase and phosphodiesterase enzymatic activities. ENPP1 cleaves a variety of substrates, including phosphodiester bonds of nucleotides and nucleotide sugars and pyrophosphate bonds of nucleotides and nucleotide sugars. ENPP1 may also hydrolyze nucleoside 5′ triphosphates to their corresponding monophosphates and may also hydrolyze diadenosine polyphosphates. Further, ENPP1 is widely expressed in several tissues and plays a role in cancers; and in cardiovascular, neurological, immunological, periodontal, musculoskeletal, hormonal, and hematological functions in mammals (Onyedibe, et al., Molecules 2019, 24, 4192). Therefore, ENPP1 inhibitors play a role in treating diseases and/or disorders associated with tissues that express ENPP1, where the disorder involves ENPP1 activity, inactivity, or signaling.

Recently, loss of function mutations that knock out Enpp1 and other genes (e.g., ANK) have been performed and the effects on cementum growth, bone growth, and bone mineralization have been investigated (Nagasaki, et al., J. Dent. Res. 2021, 100(6): 639-647). While the studies showed promotion of cementogenesis in Enpp1 knockouts compared to control, a similar regenerative response was not observed in bone (Nagasaki, et al., J. Dent. Res. 2021, 100(6): 639-647). Investigations have also observed (i) reduced femur length and increased ectopic calcifications in Ank, Enpp1 double knockouts compared to single knockout mice, and (ii) exacerbation of osteopenia in adult Enpp1 knockout mice (Chu, et al., Bone 2020, 136, 115329; Harmey, et al., Am. J. Pathol. 2004, 164(4), 1199-1209; Mackenzie, et al., PLoS One 2012, 7(2):e32177; Nagasaki, et al., J. Dent. Res. 2021, 100(6): 639-647). It is noteworthy, that these studies did not demonstrate bone growth differences across genotypes and in some instances reduced bone (femur) length. Accordingly, there remains a need to develop other approaches to promote bone growth, bone mineralization, or both.

Therefore, it is an object of the present invention to provide compositions that promote bone growth, bone mineralization, or both.

It is another object of the present invention to provide compositions that that contain ectonucleotide pyrophosphatase/phosphodiesterase 1 (ENPP1) inhibitors in effective amounts to promote bone growth, bone mineralization, or both.

It is another object of the present invention to provide compositions that that contain ENPP1 inhibitors in effective amounts to promote alveolar bone growth, alveolar bone mineralization, or both.

It is yet another object of the present invention to provide methods of using the compositions containing effective amounts of ENPP1 inhibitors to promote bone growth, bone mineralization, or both, in disorders that exhibit bone loss and/or reduced bone density.

### SUMMARY OF THE INVENTION

Compositions containing (a) an ENPP1 inhibitor or a pharmaceutically acceptable salt thereof in an effective amount to promote bone mineralization, bone growth, or both, and (b) a pharmaceutically acceptable excipient have been developed. The disclosed ENPP1 inhibitors are widely applicable in the general process of bone growth, bone mineralization, or both.

In some forms, the ENPP1 inhibitor binds to the extra-cellular domain of ENPP1 containing an active site of ENPP1, with two Zn²⁺ ions. In some forms, the ENPP1 inhibitor is a non-nucleoside-based ENPP1 inhibitor that has a structural similarity of between 0.85 and 1.0 to the structure of N-[[4-(7-methoxy-4-quinolinyl)phenyl]methyl]-sulfamide, as measured using a Tanimoto coefficient with molecular descriptors selected from two-dimensional molecular fingerprints. In some forms, the ENPP1 inhibitor is N-[[4-(7-methoxy-4-quinolinyl)phenyl]methyl]-sulfamide.

The compositions can be administered via one or more routes of administration. Exemplary routes of administration are topical, mucosal, buccal, transdermal, intradermal, intravenous, intramuscular, intra-articular, intraperitoneal, oral, intrathecal, intraspinal, intranasal, intracranial, or combinations thereof. Preferably, the compositions are administered topically, mucosally, buccally, transdermally, intradermally, intramuscularly, intra-articularly, intraspinally, or combinations thereof.

### BRIEF DESCRIPTION OF THE DRAWINGS

FIGS. 1A-1D are bar graphs showing alveolar bone response to different treatments. FIG. 1A demonstrates variability of mandible size. FIG. 1B shows alveolar bone volume on the fenestration side compared to unoperated side.

FIG. 1C shows the calculated change in volume (fenestration volume—unoperated volume) to account for size variability. FIG. 1D shows percent change in alveolar bone relative to dimethyl sulfoxide (DMSO) treatment group.

FIGS. 2A-2D are bar graphs showing dentin/cementum response to different treatments. FIG. 2A demonstrates variability of mandibular first molars. FIG. 2B shows dentin/cementum volume on the fenestration side compared to unoperated side. FIG. 2C shows the calculated change in volume (fenestration volume—unoperated volume) to account for size variability in teeth. This controls for areas of the tooth that should not be different between unoperated and fenestration defect side (e.g., tooth crown). FIG. 2D shows percent change in dentin/cementum relative to DMSO treatment group.

### DETAILED DESCRIPTION OF THE INVENTION

### I. Definitions

“About,” as relates to a numerical values, refers to variations of 10% of the specified numerical values.

“Lipinski's rule of five” is a rule of thumb for determining the bioavailability of orally administered drugs. The rule indicates that drug with good bioavailability, post-oral administration, general have no more than five hydrogen bond donors, no more than 10 hydrogen bond acceptors, a molecular weight less than 500 Da, and an octanol-water partition coefficient of no more than 5.

“Nucleoside-based,” as relates to ENPP1 inhibitors, refers to ENPP1 inhibitors that contain a nucleobase covalently bonded directly or indirectly to a ribose or deoxyribose monosaccharide. The nucleobase is cytosine, guanine, adenine, thymine, and adenine.

“Non-nucleoside-based,” as relates to ENPP1 inhibitors, refers to ENPP1 inhibitors that do not contain a nucleobase covalently bonded directly or indirectly to a ribose or deoxyribose monosaccharide.

“Pharmaceutically acceptable salt” refers to the modification of the original compound by making the acid or base salts thereof. Examples of pharmaceutically acceptable salts include, but are not limited to, mineral or organic acid salts of basic residues such as amines and alkali or organic salts of acidic residues such as carboxylic acids. For original compounds containing a basic residue, pharmaceutically acceptable salts can be prepared by treating the compounds with an appropriate amount of a non-toxic inorganic or organic acid. Suitable inorganic acids include hydrochloric, hydrobromic, sulfuric, sulfamic, phosphoric, and nitric acids; suitable organic acids include acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic acids. For original compounds containing an acidic residue, pharmaceutically acceptable salts can be prepared by treating the compounds with an appropriate amount of a non-toxic base. Suitable non-toxic bases include ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, and histidine. Generally, pharmaceutically acceptable salts can be prepared by reacting the free acid or base form of the original compounds with a stoichiometric amount of the appropriate base or acid, respectively, in water or in an organic solvent, or in a mixture thereof.

Non-aqueous media like ether, ethyl acetate, ethanol, isopropanol, acetonitrile, or combinations thereof can be used. Lists of suitable pharmaceutically acceptable salts can be found in Remington's Pharmaceutical Sciences, 20th Ed., Lippincott Williams & Wilkins, Baltimore, M D, 2000, p. 704; and Handbook of Pharmaceutical Salts: Properties, Selection, and Use, Stahl and Wermuth, Eds., Wiley-VCH, Weinheim, 2002.

“Small molecule” refers to a molecule having a molecular weight less than 2,500 Da, such as between 200 Da and 2,500 Da.

The terms “treatment” and “treating” refer to the medical management of a subject with the intent to cure, ameliorate, stabilize, or prevent one or more symptoms of a disease or disorder. This term includes active treatment toward the improvement of a disease or disorder. In addition, this term includes palliative treatment, that is, treatment designed for the relief of symptoms rather than the curing of the disease or disorder; preventative treatment, that is, treatment directed to minimizing or partially or completely inhibiting the development of the associated disease or disorder; and supportive treatment, that is, treatment employed to supplement another specific therapy directed toward the improvement of the associated disease or disorder. It is understood that treatment, while intended to cure, ameliorate, stabilize, or prevent a disease or disorder, need not actually result in the cure, amelioration, stabilization or prevention. The effects of treatment can be measured or assessed as described herein and as known in the art as is suitable for the disease or disorder involved. Such measurements and assessments can be made in qualitative and/or quantitative terms. Thus, for example, characteristics or features of a disease or disorder and/or symptoms of a disease or disorder can be reduced to any effect or to any amount.

### II. Compositions

Disclosed are compositions containing (a) an ENPP1 inhibitor or a pharmaceutically acceptable salt thereof in an effective amount to promote bone mineralization, bone growth, or both, and (b) a pharmaceutically acceptable excipient. It has been discovered that inhibition of ENPP1 using molecular inhibitors promotes bone growth, such as alveolar bone growth. Given the significant similarities of the cells and pathways involved in bone growth, the disclosed ENPP1 inhibitors are widely applicable in the general process of bone growth, bone mineralization, or both.

In some forms, the ENPP1 inhibitor binds to the extra-cellular domain of ENPP1. In some forms, the ENPP1 inhibitor binds to an active site of ENPP1, containing one or more (such as two) cations (such as Zn²⁺). Preferably, the compound inhibits ENPP1 activity. The ENPP1 activity includes, but is not limited to, cleaving phosphodiester bonds of nucleotides and nucleotide sugars and pyrophosphate bonds of nucleotides and nucleotide sugars, hydrolysis of nucleoside 5′ triphosphates to their corresponding monophosphates, and hydrolysis of diadenosine polyphosphates.

In some forms, the ENPP1 inhibitor is a small molecule. In some forms, the ENPP1 inhibitor is a non-nucleoside-based or a nucleoside-based inhibitor. In some forms, the ENPP1 inhibitor has a structural similarity of between 0.5 and 1.0, between 0.7 and 1.0, or between 0.85 and 1.0 to the structure of N-[[4-(7-methoxy-4-quinolinyl)phenyl]methyl]-sulfamide, as measured using a Tanimoto coefficient with molecular descriptors selected from two-dimensional molecular fingerprints, two-dimensional topological indices, two-dimensional maximum common substructures, three-dimensional overall shape, and three-dimensional molecular fields. In some forms, the ENPP1 inhibitor is N-[[4-(7-methoxy-4-quinolinyl)phenyl]methyl]-sulfamide.

In some forms, the ENPP1 inhibitor has a topological polar surface area (i) between 70 Å and 140 Å, or (ii) greater than 140 Å. In some forms, the ENPP1 inhibitor has a molecular weight (i) between 200 Da and 500 Da, or (ii) greater than 500 Da and no more than 2,500 Da. In some forms, the ENPP1 inhibitor has one or more of hydrogen bond donors, hydrogen bond acceptors, molecular weight, and octanol-water partition coefficient non-conforming with Lipinski's rule of five.

In some forms, the ENPP1 inhibitor is:
(i) in a solution;
(ii) in a suspension;
(iii) in a gel; or
(iv) encapsulated and/or bound to an implant, nanoparticle, microparticle, nanogel, microgel.

Additional examples of ENPP1 inhibitors are described in U.S. Pat. No. 10,689,376 to Vankayalapati, et al.; WO2019/104316 by Somerman, et al.; WO2021/257614 by Cogan, et al., Carozza, et al.; WO2021/225969 by Cogan, et al., Carozza, et al.; Cell Chemical Biology 2020, 27, 1-12; Gangar, et al., Bioorg. Chem. 2022, 119, 105549; Onyedibe, et al., Molecules 2019, 24, 4192; Patel, et al., Bioorg. Med. Chem. Lett. 2009, 19, 3339-3343; WO2022/056068 by Deb, et al., or U.S. Patent Application Publication 2021/0369747 by Li, et al. The contents of these documents are herein incorporated in their entirety, by reference.

### III. Methods of Making and Reagents Therefor

The compounds in the methods and compositions described herein can be synthesized using methods known to those of skill in the art of organic chemistry synthesis. In some forms, some of the compounds can be purchased from one or more commercial vendors.

### IV. Methods of Using

As noted above, significant similarities exist in the cells and pathways involved in bone growth. Therefore, the disclosed ENPP1 inhibitors are widely applicable in the general process of bone growth, bone mineralization, or both.

The methods typically include administering to a subject in need thereof a disclosed composition or formulation containing an effective amount of an ENPP1 inhibitor or a pharmaceutically acceptable salt thereof, to promote bone growth and/or bone mineralization in the subject. The precise dosage will vary according to a variety of factors such as subject-dependent variables (such as age, immune system health, etc.), the disease, disorder, and the treatment being effected.

Several diseases or disorders that can lead to bone loss and/or reduced bone mineralization are known, and in particular bone disorders that are driven by inflammation (Redlich, et al., Nat. Rev. Drug Discov. 2012, 11(3), 234-50). The classes of these diseases or disorders include periodontal disease; autoimmune disorders; inflammatory disorders; metabolic disorder; digestive and gastrointestinal disorders; side effects from medical procedures; cancer; hematologic/blood disorders; neurological/nervous system disorders; bone marrow disorders; endocrine disorders; ageing; and combinations thereof. Specific examples of these diseases or disorders include, but are not limited to, periodontal disease, rheumatoid arthritis; lupus; multiple sclerosis; ankylosing spondylitis; celiac disease; inflammatory bowel disease; side effects from weight loss surgery, gastrectomy, and gastrointestinal bypass procedures; cancer; leukemia; lymphoma; multiple myeloma; sickle cell disease; stroke; Parkinson's disease; multiple sclerosis; vertebral column injuries; thalasemia; diabetes; hyperparathyroidism; hyperthyroidism; Cushing's syndrome; thyrotoxicosis; irregular periods; premature menopause; low levels of testosterone and estrogen in men; and combinations thereof. As such, the disclosed compositions are useful in promoting bone growth and/or bone mineralization incidental to these diseases or disorders. The compositions can also be used to promote bone growth and/or bone mineralization due to ageing.

In some forms, compositions can be used to promote bone growth and/or bone mineralization in a subject suffering from a periodontal disease. In some the compositions can be used to promote bone mineralization, bone growth, or both, of an alveolar bone.

In some forms, the effective amount of the ENPP1 inhibitor or a pharmaceutically acceptable salt thereof, which promote bone growth and/or bone mineralization reduces ENPP1 signaling and/or enzymatic activity. In some forms, the effective amount reduces nucleotide and/or nucleotide binding to ENPP1. In some forms, the effective amount reduces activation of an ENPP1 pathway. The activity may include modulating phosphodiester bond hydrolysis, pyrophosphate bond hydrolysis, or a combination thereof. In some forms, the activity may include inhibiting cyclic guanosine monophosphate-adenosine monophosphate (cGAMP) hydrolysis, nucleoside 5′ triphosphate hydrolysis (such as ATP hydrolysis), diadenosine polyphosphate hydrolysis, or a combination thereof.

The effective amount of the ENPP1 inhibitor can be ascertained from assays investigating the inhibition of ENPP1-nucleotide/nucleotide binding compared to a control that does not contain the compound, as determined by an assay that detects fluorescence polarization. In some forms, the effective amount of the ENPP1 inhibitor is greater than about 5 μM, 7.5 μM, or 10 μM. In some forms, the effective amount of the ENPP1 inhibitor is between about 5 μM and about 10,000 μM, between about 7.5 μM and about 10,000 μM, between about 10 μM and about 10,000 μM, between about 5 μM and about 1,000 μM, between about 7.5 μM and about 1,000 μM, or between about 10 μM and about 1,000 μM, between about 5 μM and about 100 μM, between about 7.5 μM and about 100 μM, or between about 10 μM and about 100 μM, or any subrange or specific number therebetween.

In some forms, the amount of the ENNP1 inhibitor administered can be greater than about 300 μg, 450 μg, or 600 μg. In some forms, the effective amount of the ENPP1 inhibitor is between about 310 μg and about 650 mg, between about 450 μg and about 650 mg, between about 600 μg and about 650 mg, between about 300 μg and about 65 mg, between about 450 μg and about 65 mg, or between about 600 μg and about 65 mg, between about 300 μg and about 6.5 mg, between about 450 μg and about 6.5 mg, or between about 600 μg and about 6.5 mg, or any subrange or specific number therebetween.

The compositions can be administered in a single dose or in multiple doses. When multiple doses are administered, the unit dosage may be the same or different for each administration. Certain factors may influence the dosage required to effectively treat a subject, including but not limited to the severity of the disease or disorder, previous treatments, the general health and/or age of the subject, and other diseases present. It will also be appreciated that the effective dosage of the composition used for treatment may increase or decrease over the course of a particular treatment. Changes in dosage may result and become apparent from the results of diagnostic assays.

Dosing is dependent on severity and responsiveness of the disease condition to be treated, and the course of treatment may last from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies, and repetition rates.

Additional Formulations

The compounds described herein can be formulated for enteral, parenteral, topical, or pulmonary administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients. See, e.g., Remington's Pharmaceutical Sciences, latest edition, by E.W. Martin Mack Pub. Co., Easton, PA, which discloses typical carriers and conventional methods of preparing pharmaceutical compositions that can be used in conjunction with the preparation of formulations of the compounds described herein and which is incorporated by reference herein. These most typically would be standard carriers for administration of compositions to humans. In one aspect, humans and non-humans, including solutions such as sterile water, saline, and buffered solutions at physiological pH. Other compounds will be administered according to standard procedures used by those skilled in the art.

These formulations can take the form of solutions, suspensions, emulsion, gel, cream, lotion, transdermal patch, oils, tablets, pills, capsules, powders, sustained-release formulations such as nanoparticles, microparticles, etc., and the like.

i. Parenteral Formulations

The compositions described herein can be formulated for parenteral administration. For example, parenteral administration may include administration to a patient intrathecally, instraspinally, intranasally, topically, mucosally, bucally, transdermally, intravenously, intradermally, intraarterially, intraperitoneally, intralesionally, intracranially, intraarticularly, intraprostatically, intrapleurally, intratracheally, intravitreally, intratumorally, intramuscularly, intra-articularly, subcutaneously, intravesicularly, intraumbilically, by injection, and by infusion.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

If for intravenous administration, the compositions are packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine, and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

If needed, the formulation can be buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

1. Controlled Release Formulations

The parenteral formulations described herein can be formulated for controlled release including immediate release, delayed release, extended release, pulsatile release, and combinations thereof.

(a) Nano- and Microparticles

For parenteral administration, the one or more compounds, and optional one or more additional active agents, can be incorporated into microparticles, nanoparticles, or combinations thereof that provide controlled release of the compounds and/or one or more additional active agents. In forms wherein the formulations contain two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

(b) Method of Making Nano- and Microparticles

Methods for preparing microparticles and nanoparticles include, but are not limited to, self-assembly; crosslinking; solvent evaporation and/or emulsion encapsulation (such as single emulsion solvent evaporation or multi-emulsion solvent evaporation); hot melt particle formation; solvent removal; spray drying; phase inversion; microfluidics; coacervation; low temperature casting; molecular dispersion or phase separated dispersion techniques; or solid phase encapsulation techniques.

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some forms, drug in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some forms, drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings and/or lacquers. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating and/or lacquer layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

2. Injectable/Implantable Formulations

The compounds described herein can be incorporated into injectable/implantable solid or semi-solid implants, such as polymeric implants. In some forms, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication requires polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods.

The release of the one or more compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

ii. Enteral Formulations

Oral formulations can include standard carriers such as pharmaceutical grades of mannitol, lactose, sodium saccharine, starch, magnesium stearate, cellulose, magnesium carbonate, etc. Such compositions will contain a therapeutically effective amount of the compound and/or antibiotic together with a suitable amount of carrier so as to provide the proper form to the patient based on the mode of administration to be used.

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating and/or lacquer composition, which may include plasticizers, pigments, colorants, stabilizing agents, and glidants.

Lacquer materials are generally known in the art, and include thermoplastic coatings that form films by solvent evaporation. These include nitrocellulose, cellulose acetate butyrate, acrylic resins, etc. Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

1. Controlled Release Enteral Formulations

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In another form, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another form, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

(a) Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred forms, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred forms, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred form, the acrylic polymer is an acrylic resin lacquer such as that which is commercially available from Rohm Pharma under the tradename EUDRAGIT®. In further preferred forms, the acrylic polymer comprises a mixture of two acrylic resin lacquers commercially available from Rohm Pharma under the tradenames EUDRAGIT® RL30D and EUDRAGIT® RS30D, respectively. EUDRAGIT® RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 are also preferred. The code designations RL (high permeability) and RS (low permeability) refer to the permeability properties of these agents. EUDRAGIT® RL/RS mixtures are insoluble in water and in digestive fluids. However, multiparticulate systems formed to include the same are swellable and permeable in aqueous solutions and digestive fluids.

The polymers described above such as EUDRAGIT® RL/RS may be mixed together in any desired ratio in order to ultimately obtain a sustained-release formulation having a desirable dissolution profile. Desirable sustained-release multiparticulate systems may be obtained, for instance, from 100% EUDRAGIT® RL, 50% EUDRAGIT® RL and 50% EUDRAGIT t® RS, and 10% EUDRAGIT® RL and 90% EUDRAGIT® RS. One skilled in the art will recognize that other acrylic polymers may also be used, such as, for example, EUDRAGIT® L.

Alternatively, extended release formulations can be prepared using osmotic systems or by applying a semi-permeable coating and/or lacquer to the dosage form. In the latter case, the desired drug release profile can be achieved by combining low permeable and high permeable coating and/or lacquer materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating and/or lacquer or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

(b) Delayed Release Dosage Forms

Delayed release formulations can be created by coating and/or lacquer a solid dosage form with a polymer film, which is insoluble in the acidic environment of the stomach, and soluble in the neutral environment of the small intestine.

The delayed release dosage units can be prepared, for example, by coating, and/or applying a lacquer to, a drug or a drug-containing composition with a selected coating and/or lacquer material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating and/or lacquer materials may also be used. Multi-layer coatings and/or lacquers using different polymers may also be applied.

The preferred coating and/or lacquer weights for particular coating and/or lacquer materials may be readily determined by those skilled in the art by evaluating individual release profiles for tablets, beads and granules prepared with different quantities of various coating and/or lacquer materials. It is the combination of materials, method and form of application that produce the desired release characteristics, which one can determine only from the clinical studies.

The coating and/or lacquer composition may include conventional additives, such as plasticizers, pigments, colorants, stabilizing agents, glidants, etc. A plasticizer is normally present to reduce the fragility of the coating and/or lacquer, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating and/or lacquer solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating and/or lacquer composition.

The disclosed compositions and methods of using can be further understood through the following enumerated paragraphs or embodiments.
1. A composition containing (a) an ENPP1 inhibitor or a pharmaceutically acceptable salt thereof in an effective amount to promote bone mineralization, bone growth, or both, and (b) a pharmaceutically acceptable excipient.
2. A composition containing:
(a) an ENPP1 inhibitor or a pharmaceutically acceptable salt thereof in a concentration,
   (i) between about 5 μM and about 10,000 μM, between about 7.5 μM and about 10,000 μM, or between about 10 μM and about 10,000 μM, or
   (ii) between about 310 μg and about 650 mg, between about 450 μg and about 650 mg, or between about 600 μg and about 650 mg, and
(b) a pharmaceutically acceptable excipient.
3. The composition of paragraph 1 or 2, wherein the ENPP1 inhibitor is a small molecule.
4. The composition of any one of paragraphs 1 to 3, wherein the ENPP1 inhibitor is a non-nucleoside-based or a nucleoside-based inhibitor.
5. The composition of any one of paragraphs 1 to 4, wherein the ENPP1 inhibitor is a non-nucleoside-based inhibitor.
6. The composition of any one of paragraphs 1 to 4, wherein the ENPP1 inhibitor is a nucleoside-based inhibitor.
7. The composition of any one of paragraphs 1 to 6, wherein the ENPP1 inhibitor has a structural similarity of between 0.5 and 1.0, between 0.7 and 1.0, or between 0.85 and 1.0 to the structure of N-[[4-(7-methoxy-4-quinolinyl)phenyl]methyl]-sulfamide, as measured using a Tanimoto coefficient with molecular descriptors selected from two-dimensional molecular fingerprints, two-dimensional topological indices, two-dimensional maximum common substructures, three-dimensional overall shape, and three-dimensional molecular fields.
8. The composition of any one of paragraphs 1 to 7, wherein the ENPP1 inhibitor is N-[[4-(7-methoxy-4-quinolinyl)phenyl]methyl]-sulfamide.
9. The composition of any one of paragraphs 1 to 8, wherein the effective amount is greater than about 5 μM, 7.5 μM, or 10 μM.
10. The composition of any one of paragraphs 1 to 9, wherein the effective amount is between about 5 μM and about 100 μM, between about 7.5 μM and about 100 μM, or between about 10 μM and about 100 μM.
11. The composition of any one of paragraphs 1 to 10, wherein the effective amount is in a volume of greater than about 1 μL.
12. The composition of any one of paragraphs 1 to 11, wherein the ENPP1 inhibitor has a topological polar surface area (i) between 70 Å and 140 Å, or (ii) greater than 140 Å.
13. The composition of any one of paragraphs 1 to 12, wherein the ENPP1 inhibitor has a molecular weight (i) between 200 Da and 500 Da, or (ii) greater than 500 Da and no more than 2,500 Da.
14. The composition of any one of paragraphs 1 to 13, wherein the ENPP1 inhibitor has one or more of hydrogen bond donors, hydrogen bond acceptors, molecular weight, and octanol-water partition coefficient non-conforming with Lipinski's rule of five.
15. The composition of any one of paragraphs 1 to 14, wherein the ENPP1 inhibitor is:
(i) in a solution;
(ii) in a suspension;
(iii) in a gel; or
(iv) encapsulated and/or bound to an implant, nanoparticle, microparticle, nanogel, microgel.
16. A method of promoting bone mineralization, bone growth, or both, in a subject in need thereof, the method comprising administering to the subject the composition of any one of paragraphs 1 to 15.
17. The method of paragraph 16, wherein the composition is administered topically, mucosally, buccally, transdermally, intradermally, intravenously, intramuscularly, intra-articularly, intraperitoneally, orally, intrathecally, intraspinally, intranasally, intracranially, or combinations thereof.
18. The method of paragraph 16 or 17, wherein the subject is suffering from bone loss, reduced bone mineralization, or both, related to disorders selected from periodontal disease; autoimmune disorders; inflammatory disorders; metabolic disorders; digestive and gastrointestinal disorders; side effects from medical procedures; cancer; hematologic/blood disorders; neurological/nervous system disorders; bone marrow disorders; endocrine disorders; ageing; and combinations thereof.
19. The method of paragraph 18, wherein the related disorders are selected from rheumatoid arthritis; lupus; multiple sclerosis; ankylosing spondylitis; celiac disease; inflammatory bowel disease; side effects from weight loss surgery, gastrectomy, and gastrointestinal bypass procedures; cancer; leukemia; lymphoma; multiple myeloma; sickle cell disease; stroke; Parkinson's disease; multiple sclerosis; vertebral column injuries; thalasemia; diabetes; hyperparathyroidism; hyperthyroidism; Cushing's syndrome; thyrotoxicosis; irregular periods; premature menopause; low levels of testosterone and estrogen in men; and combinations thereof.
20. The method of any one of paragraphs 16 to 19, wherein the subject is suffering from a periodontal disease.
21. The method of any one of paragraphs 16 to 20, wherein the composition is administered to promote bone mineralization, bone growth, or both, of an alveolar bone.

### EXAMPLES

### Example 1: Inhibition of ENPP1 and Bone Growth

### Materials and Methods

Fenestration defects were created on buccal aspect of mandibular first molar, distal root, in 6-7 week old mice. Collagen sponge with 1 uL of either alkaline phosphatase (ALPL) (100 ng/uL), DMSO (1 uL undiluted), high dose ENPP1 inhibitor (10 uM), low dose ENPP1 inhibitor (1 uM) was applied in the defect (n=3 or 4 for each group). Mice were euthanized 28 days later, and their mandibles were microCT scanned (unoperated side, defect side). A region of interest was defined as follows:
Right and left sides registered to each other to standardize position;
480 microns mesial to and 3600 microns distal to first molar mesial height of contour; and

Region was selected to encompass area that exhibited regenerated bone. Unoperated side with defect side were registered, the enamel was removed from analysis, and change in volume (defect side—control side) was calculated from for dentin/cementum and alveolar bone for each treatment: ALPL, DMSO, low dose ENPP1 inhibitor, and high dose ENPP1 inhibitor. Regenerated bone area was much larger than defect size. To make the defect, muscle attachment was severed, and the region was curetted. Because the collagen sponge was placed in the defect and not fully enclosed (muscle was repositioned and sutured over sponge), there could be some leakage of DMSO/ALP/ENPP1 inhibitor, further stimulating bone growth.

### Results

The mice were 6-7 weeks old at time of surgery. Therefore, crown and root dentin developments were complete, such that change in tooth volume was not attributed to developmental stages in the mice. Variability affected statistical significance, and may require additional animals per group, e.g., there was one animal in the high dose group that did not have much change between defect side and unoperated side.

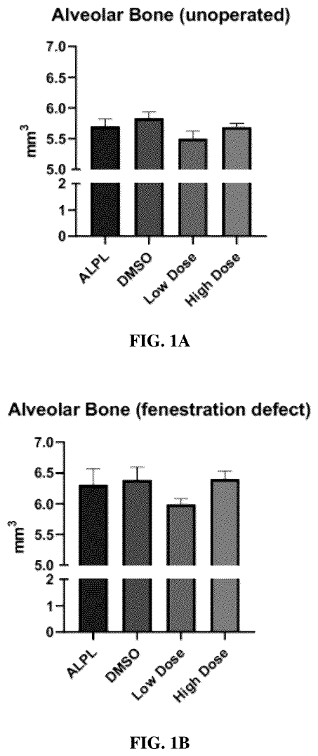

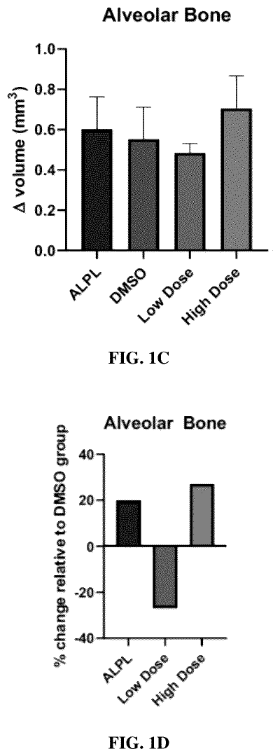

The microCT results for bone are shown in FIGS. 1A-1D. As shown in FIG. 1A, the low dose group were in general smaller compared to the other groups. In all groups alveolar bone volume was higher on the fenestration side compared to unoperated side (FIG. 1B).

Density heat maps were generated to visualize cementum. The heat maps indicated regeneration of cementum. On the fenestration side, bone densities also appear higher in high dose, compared to ALPL, DMSO, and low dose.

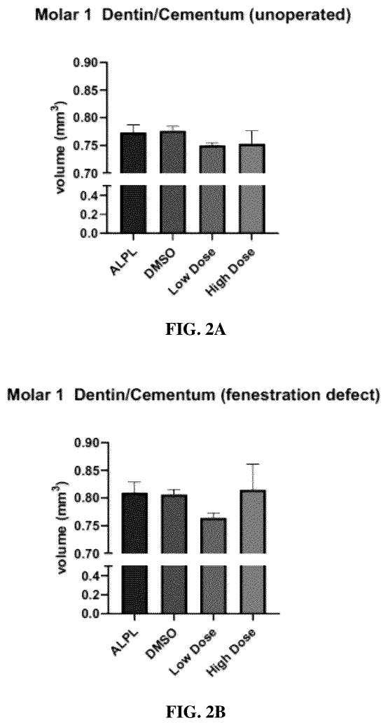

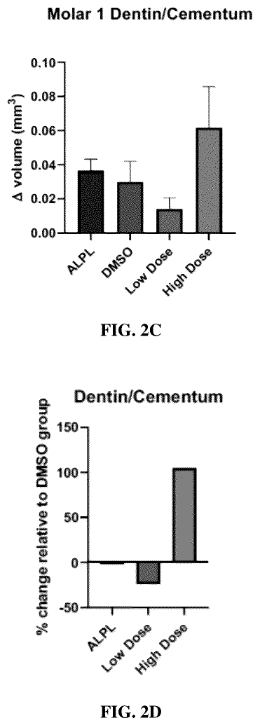

The microCT results for dentin/cementum are shown in FIGS. 2A-2D. The low dose group were in general smaller compared to the other groups (FIG. 2A). In all groups the mandibular first molar on the fenestration side had higher volumes compared to unoperated side (FIG. 2B). Not shown is the second mandibular that display no change in volume.

Unless defined otherwise, all technical and scientific terms used herein have the same meanings as commonly understood by one of skill in the art to which the disclosed invention belongs. Publications cited herein and the materials for which they are cited are specifically incorporated by reference. Further, unless otherwise indicated, use of the expression “wt %” refers to “wt/wt %.”

Those skilled in the art will recognize, or be able to ascertain using no more than routine experimentation, many equivalents to the specific embodiments of the invention described herein. Such equivalents are intended to be encompassed by the following claims.

I claim:

1. A composition comprising (a) an ENPP1 inhibitor or a pharmaceutically acceptable salt thereof in an effective amount to promote bone mineralization, bone growth, or both, and (b) a pharmaceutically acceptable excipient.

2. A composition comprising:
(a) an ENPP1 inhibitor or a pharmaceutically acceptable salt thereof in a concentration,
(i) between about 5 μM and about 10,000 μM, between about 7.5 μM and about 10,000 μM, or between about 10 μM and about 10,000 μM, or
(ii) between about 310 μg and about 650 mg, between about 450 μg and about 650 mg, or between about 600 μg and about 650 mg, and
(b) a pharmaceutically acceptable excipient.

3. The composition of claim 1, wherein the ENPP1 inhibitor is a small molecule.

4. The composition of claim 1, wherein the ENPP1 inhibitor is a non-nucleoside-based or a nucleoside-based inhibitor.

5. The composition of claim 1, wherein the ENPP1 inhibitor is a non-nucleoside-based inhibitor.

6. The composition of claim 1, wherein the ENPP1 inhibitor is a nucleoside-based inhibitor.

7. The composition of claim 1, wherein the ENPP1 inhibitor has a structural similarity of between 0.5 and 1.0, between 0.7 and 1.0, or between 0.85 and 1.0 to the structure of N-[[4-(7-methoxy-4-quinolinyl)phenyl]methyl]-sulfamide, as measured using a Tanimoto coefficient with molecular descriptors selected from two-dimensional molecular fingerprints, two-dimensional topological indices, two-dimensional maximum common substructures, three-dimensional overall shape, and three-dimensional molecular fields.

8. The composition of claim 1, wherein the ENPP1 inhibitor is N-[[4-(7-methoxy-4-quinolinyl)phenyl]methyl]-sulfamide.

9. The composition of claim 1, wherein the effective amount is greater than about 5 μM, 7.5 μM, or 10 μM.

10. The composition of claim 1, wherein the effective amount is between about 5 μM and about 100 μM, between about 7.5 μM and about 100 PM, or between about 10 μM and about 100 μM.

11. The composition of claim 1, wherein the effective amount is in a volume of greater than about 1 μL.

12. The composition of claim 1, wherein the ENPP1 inhibitor has a topological polar surface area (i) between 70 Å and 140 Å, or (ii) greater than 140 Å.

13. The composition of claim 1, wherein the ENPP1 inhibitor has a molecular weight (i) between 200 Da and 500 Da, or (ii) greater than 500 Da and no more than 2,500 Da.

14. The composition of claim 1, wherein the ENPP1 inhibitor has one or more of hydrogen bond donors, hydrogen bond acceptors, molecular weight, and octanol-water partition coefficient non-conforming with Lipinski's rule of five.

15. The composition of claim 1, wherein the ENPP1 inhibitor is:
(i) in a solution;
(ii) in a suspension;
(iii) in a gel; or
(iv) encapsulated and/or bound to an implant, nanoparticle, microparticle, nanogel, microgel.

16. A method of promoting bone mineralization, bone growth, or both, in a subject in need thereof, the method comprising administering to the subject the composition of claim 1.

17. The method of claim 16, wherein the composition is administered topically, mucosally, buccally, transdermally, intradermally, intravenously, intramuscularly, intra-articularly, intraperitoneally, orally, intrathecally, intraspinally, intranasally, intracranially, or combinations thereof.

18. The method of claim 16, wherein the subject is suffering from bone loss, reduced bone mineralization, or both, related to disorders selected from periodontal disease; autoimmune disorders; inflammatory disorders; metabolic disorders; digestive and gastrointestinal disorders; side effects from medical procedures; cancer; hematologic/blood disorders; neurological/nervous system disorders; bone marrow disorders; endocrine disorders; ageing; and combinations thereof.

19. The method of claim 18, wherein the related disorders are selected from rheumatoid arthritis; lupus; multiple sclerosis; ankylosing spondylitis; celiac disease; inflammatory bowel disease; side effects from weight loss surgery, gastrectomy, and gastrointestinal bypass procedures; cancer; leukemia; lymphoma; multiple myeloma; sickle cell disease; stroke; Parkinson's disease; multiple sclerosis; vertebral column injuries; thalasemia; diabetes; hyperparathyroidism; hyperthyroidism; Cushing's syndrome; thyrotoxicosis; irregular periods; premature menopause; low levels of testosterone and estrogen in men; and combinations thereof.

20. The method of claim 16, wherein the subject is suffering from a periodontal disease.

21. The method of claim 16, wherein the composition is administered to promote bone mineralization, bone growth, or both, of an alveolar bone.
